(19) United States
(12) Patent Application Publication
(10) Pub. No.: US 2024/0148720 A1
(43) Pub. Date: May 9, 2024

(54) Use of Histamine Antagonists and Glucocorticoid for Treating and Preventing Neurodegenerative Diseases

(71) Applicant: City University of Hong Kong, Kowloon (HK)

(72) Inventors: Chi Him Eddie Ma, Kowloon (HK); Yi Jun Xu, Kowloon (HK)

(21) Appl. No.: 18/452,885

(22) Filed: Aug. 21, 2023

Related U.S. Application Data
(60) Provisional application No. 63/380,404, filed on Oct. 21, 2022.

(51) Int. Cl. A61K 31/496; A61K 31/573; A61P 25/28

(52) U.S. Cl. CPC A61K 31/496; A61K 31/573; A61P 25/28

(57) ABSTRACT

A method for treating and preventing neurodegenerative diseases, a method of reversing cognitive and memory deficit, a method of decreasing Aβ level and hyperphosphorylated Tau in a subject in need thereof with histamine antagonists, glucocorticoid, or their combinations includes the step of administrating a histamine H4 receptor antagonist or a glucocorticoid, or their combination to the subject.

The current application claims a priority to the U.S. Provisional Patent application Ser. No. 63/380,404 filed on Oct. 21, 2022.

### FIELD OF THE INVENTION

The present invention generally relates to use of histamine antagonists and glucocorticoid for treating neurodegenerative diseases.

### BACKGROUND

Neurodegenerative diseases affect millions of people worldwide. Alzheimer's Disease (AD) and Parkinson's Disease (PD) are the most common neurodegenerative diseases, while Alzheimer's disease is the most common form of dementia. AD is a progressive neurodegenerative disease characterized by cognitive deficits, memory impairment, and behavior disorder. Currently, there are more than 40 million AD patients in the world, and the number will increase to 130 million in 2050. Due to the life-long treatment for this progressive disease, if there is no effective drug that can stop or reverse the disease progression, the global cost of treatment and care for AD patients is estimated to be over $1 trillion USD in 2050, and its treatment places a heavy financial and mental burden on patients' families and societies. Aβ deposition and hyperphosphorylated Tau are the major hallmarks of AD pathology leading to a massive loss of synaptic connections and neurons which contribute to memory deficits. Accumulating evidence shows that reducing Aβ plaque and hyperphosphorylated Tau accumulation could ameliorate AD symptoms.

Currently, the seven FDA-approved AD drugs, including tacrine, rivastigmine, galantamine, donepezil, Memantine, Aducanumab and Leqembi can only reduce the speed of cognitive function decline or transiently improve AD symptoms, and do not stop or reverse the disease progression in patients. While aducanumab and Leqembi might have mild beneficial effects on patients with the early stage of AD, it shows no improvement in advanced AD patients with massive Aβ plaque and widespread neuroinflammation (microgliosis) in the affected brain.

In addition, Aβ is the dominant target in the clinical trial against AD, however, the current antibody that clears the Aβ or the beta-site amyloid precursor cleaving enzyme-1 (BACE-1) inhibitors that inhibit the generation of the Aβ show a limited effect in improving the cognitive function of the AD patients. Since the high failure rate of anti-amyloid therapies, alternative strategies for drug development against AD shall be considered.

According, there remains a need for new drugs for effective therapy and/or prophylaxis of neurodegenerative diseases such as AD and PD.

### SUMMARY OF THE INVENTION

An embodiment of the present invention relates to a method of therapy and/or prophylaxis of a neurodegenerative disease in a subject in need thereof, including administrating a histamine H4 receptor antagonist or a glucocorticoid, or their combination to the subject.

An embodiment of the present invention relates to a method of reversing cognitive and memory deficits in a subject in need thereof, including administrating a histamine H4 receptor antagonist or a glucocorticoid, or their combination to the subject.

An embodiment of the present invention relates to a method of decreasing Aβ level and hyperphosphorylated Tau in a subject in need thereof, including administrating a histamine H4 receptor antagonist to the subject.

An embodiment of the present invention relates to a method of inhibiting microgliosis in hippocampus in a subject in need thereof, including administrating a histamine H4 receptor antagonist or a glucocorticoid, or their combination to the subject.

An embodiment of the present invention relates to a method of decreasing Aβ level in a subject in need thereof, including administrating a glucocorticoid to the subject.

The present invention utilizes microglia in our immune system to phagocytize Aβ as an alternative method for treating neurodegenerative diseases such as AD. Without intending to be bound by theory, it is believed that histamine H4 receptor antagonist (e.g., 1-[(5-chloro-1H-benzimidazol-2-yl)carbonyl]-4-methylpiperazine maleate, also referred to as VUF6002 or JNJ10191584) can effectively decrease Aβ level and hyperphosphorylated Tau in a subject and meanwhile inhibit microgliosis in hippocampus. Therefore, it is believed that the new methods provided herein offer more comprehensive beneficial effect to treat patients having neurodegenerative disease such as AD, possibly via immunomodulatory effect.

### BRIEF DESCRIPTION OF THE DRAWINGS

FIG. 1A shows an embodiment of a treatment scheme for adult C57BL/6 mice with streptozocin-induced AD symptoms;

FIG. 1B shows an embodiment of assessing short-term spatial working memory by Y-maze spontaneous alternation test;

FIG. 1C shows an embodiment of assessing short-term recognition memory by novel object recognition test;

FIG. 1D shows an embodiment of assessing long-term spatial memory by Barnes maze test at day 25 post injection;

FIG. 1E is a graph showing the mice's error times observed in an exemplary probe test;

FIG. 1F is a graph showing the time the mice spent in the target quadrant in an exemplary probe test;

FIG. 2A shows an embodiment of a treatment scheme for APP/PS1 mice;

FIG. 2B shows an embodiment of assessing short-term spatial memory by Y-maze test;

FIG. 2C shows an embodiment of assessing short-term recognition memory by novel recognition test;

FIG. 2D shows an embodiment of assessing long-term spatial memory by water maze test;

FIG. 2E is a graph showing the number of platform crosses in an exemplary probe test where the platform is removed;

FIG. 2F is a graph showing the time the mice spent in the target quadrant in an exemplary probe test where the platform is removed;

FIG. 3A shows an embodiment of a treatment scheme for 3xTg-AD mice;

FIG. 3B shows an embodiment of assessing short-term spatial memory by Y-maze test;

FIG. 3C shows an embodiment of assessing short-term recognition memory by Novel recognition test;

FIG. 3D shows an embodiment of assessing long-term spatial memory by Barnes maze test.

FIG. 3E is a graph showing the primary error times observed in an exemplary probe test where the escape box is removed;

FIG. 3F is a graph showing the time the mice spent in the target quadrant in an exemplary probe test where the escape box is removed;

FIG. 4A shows images of the immunoreactivity of anti-6E10 (both soluble and insoluble form of Aβ) in the CA1 region of hippocampus from vehicle-treated 3xTg-AD mice; VUF6002 treated mice and the control group (Scale bar: 100 um);

FIG. 4B shows an embodiment of quantification of 6E10 fluorescence intensity performed at CA1 region of hippocampus using NIS-Elements software;

FIG. 4C shows representative photomicrographs of hippocampus immunostained with anti-AT 8 antibodies;

FIG. 4D is a graph showing quantification of AT8 fluorescence intensity performed at CA1 region using NIS-Elements software;

FIG. 5A are images showing activation of microglia in the hippocampal CA1 region of mice;

FIG. 5B is a graph showing quantification of Iba-1 fluorescence intensity performed on the hippocampal CA1 region of mice;

FIG. 6A shows an embodiment of assessing short-term spatial memory of the 8-month-mice in a Y-maze test;

FIG. 6B shows an embodiment of assessing recognition index revealed by the percentage of time in exploring the novel object in the novel object recognition test;

FIG. 6C shows an embodiment of assessing long-term spatial memory revealed by the escape latency in the water maze training trial;

FIG. 6D a graph showing the number of platform crosses in an exemplary probe test where the platform is removed;

FIG. 6E is a graph showing the time the mice spent in the target quadrant in an exemplary probe test where the platform is removed;

FIG. 7A are images showing soluble and insoluble forms of the Aβ immunostained by anti-6E10 antibody in the brain of the 8-month-old mice of different groups;

FIG. 7B shows an embodiment of quantification of the number of Aβ plaques in different treatment groups (Mean±SEM. * P<0.05; ** P<0.01; One-way ANOVA followed by Tukey post hoc test);

FIG. 7C are images showing hyperphosphorylated Tau immunostained by anti-AT8 antibody in the brain of the 17-month-old 3xTg-AD mice (n=4), and 3xTg-AD with dexamethasone treatment (n=4); and

FIG. 7D shows an embodiment of quantification of AT8 intensity in different groups with or without dexamethasone treatment (Mean±SEM. * P<0.05; ** P<0.01; T-test).

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

As used herein, “Aβ level” includes the soluble and insoluble Aβ.

As used herein, “Aβ plaques” only means insoluble Aβ.

An embodiment of the present invention relates to a method of therapy and/or prophylaxis of a neurodegenerative disease in a subject in need thereof, including administrating a histamine H4 receptor antagonist or a glucocorticoid, or their combination to the subject.

An embodiment of the present invention relates to a method of reversing cognitive and memory deficits in a subject in need thereof, including administrating a histamine H4 receptor antagonist or a glucocorticoid, or their combination to the subject.

An embodiment of the present invention relates to a method of decreasing Aβ level and hyperphosphorylated Tau in a subject in need thereof, including administrating a histamine H4 receptor antagonist to the subject.

An embodiment of the present invention relates to a method of inhibiting microgliosis in hippocampus in a subject in need thereof, including administrating a histamine H4 receptor antagonist or a glucocorticoid, or their combination to the subject.

An embodiment of the present invention relates to a method of decreasing Aβ level in a subject in need thereof, including administrating a glucocorticoid to the subject.

The present inventors surprisingly found that there is a therapeutic effect when mouse models with neurodegenerative diseases such as AD are treated with a histamine H4 receptor antagonist (e.g., VUF6002, also known as JNJ10191584, and JNJ7777120). VUF6002 is a selective H4 receptor antagonist having the structure of Formula I; JNJ7777120 is also a selective H4 receptor antagonist having the structure of Formula II:

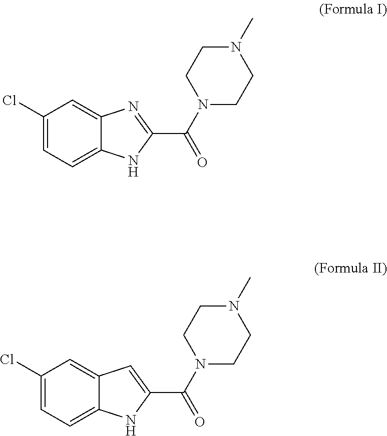 [SMILES: CN1CCN(C(=O)c2nc3cc(Cl)ccc3[nH]2)CC1 ; CN1CCN(C(=O)c2cc3cc(Cl)ccc3[nH]2)CC1]

In particular, the present invention proves that the histamine H4 receptor antagonist (including VUF6002) inhibits the activation of microglia in vivo. Without intending to be bound by theory, it is believed that reducing the number of activated microglia may help to decrease the levels of pro-inflammatory cytokines and protect the neurons from apoptosis. Besides, it is believed that histamine H4 receptor antagonists such as VUF6002 could facilitate the clearance of both the Aβ and hyperphosphorylated Tau, and inhibit neuroinflammation. Therefore, it is believed that VUF6002 has a more comprehensive beneficial effect to treat AD patients, possibly via its immunomodulatory effect. It is also believed that only a handful of the antiinflammation drugs are targeted at promoting Aβ clearance by microglia. Surprisingly, it is found that a glucocorticoid such as dexamethasone can reduce the Aβ level, hyperphosphorylated Tau levels, and improve memory deficits in the AD mice model. In particular, we are the first to report the therapeutic effect of dexamethasone (also known as MK-125; Chemical Name: (11β,16α)-9-Fluoro-11,17,21-trihydroxy-16-methylpregna-1,4-diene-3,20-dione, 9α-Fluoro-16α-methyl-11(3,17α,21-trihydroxy-1,4-pregnadiene-3,20-dione, 9α-Fluoro-16α-methylprednisolone) in the treating of an AD mouse model.

In an embodiment herein, the histamine H4 receptor antagonist is selected from the group consisting of 1-[(5-chloro-1H-benzimidazol-2-yl)carbonyl]-4-methylpiperazine maleate (VUF6002, Formula I), 1-[(5-chloro-1H-indol-2-yl)carbonyl]-4-methylpiperazine (JNJ7777120, Formula II), or a pharmaceutically acceptable salt or derivate thereof. Unlike the other histamine H4 receptor antagonists that might potentially bind to other histamine receptors (e.g. Mianserin can bind to histamine H1, H3, and H4 receptors, and Thioperamide can bind to histamine H3 and H4 receptors), VUF6002 is a potent and selective H4 receptor antagonist that shows significantly-better selectivity (>100 fold) for the H4 receptor than for the H1, H2, H3 receptors. Additionally, some histamine H4 receptor antagonists are commercially discontinued due to severe side effects, such as the JNJ39758979 which may induces agranulocytosis in humans. In contrast, VUF6002 has not been reported to have a severe side effect on the animal models.

In an embodiment herein, the neurodegenerative disease is selected from the group consisting of Alzheimer's disease, Parkinson's disease, ischemic stroke, amyotrophic lateral sclerosis, and a combination thereof. Without intending to be bound by theory, it is believed that neuroinflammation is a key pathologic marker for neurodegenerative diseases such as Alzheimer's Disease, Parkinson's Disease, ischemic stroke, and amyotrophic lateral sclerosis. Since the histamine H4 receptor antagonists can migrate the neuroinflammation, it is believed that these neurodegenerative diseases may be treated by histamine H4 receptor antagonists.

In an embodiment herein, the histamine H4 receptor antagonist is administrated at a dosage of about 0.01 mg/kg to about16 mg/kg on a daily basis. It is found that VUF6002 at about 2 mg/kg can effectively act on the histamine H4 receptors in mice. Since the larger animals have lower metabolic rates, the metabolic rates of the mouse are around 12.3 times of humans. Accordingly, the effective dosage of VUF6002 in humans is expected to be around 0.2 mg/kg by intraperitoneal injection. Intracerebroventricular administration may needs a smaller dosage of about 0.01 mg/kg.

Additionally, VUF6002 at 100 mg/kg revealed an anti-inflammation effect on rats, the metabolic rates of the rat are around 6.2 times of humans, indicating VUF6002 at about 16 mg/kg may show an anti-inflammation effect on humans. Taken together, in an embodiment herein, VUF6002 levels ranging from about 0.001 mg/kg to about 16 mg/kg; or from about 0.005 mg/kg to about 12 mg/kg; or from about 0.01 mg/kg to about 10 mg/kg in humans.

It is worth noting that the cost of VUF6002 is much lower than Aducanumab, the only FDA-approval drug to treat the mild stage of AD patients with an annual cost of about 519000 US dollars, while treating an AD patient in 60 kg with VUF6002 at 16 mg/kg on a daily basis for a year only costs about 3600 US dollars.

In an embodiment herein, the histamine H4 receptor antagonist is administrated for from about 21 days to about 3 months, for example, for about 1 month, for about 2 months, or for about 45 days, or life-long for human.

In an embodiment herein, the treatment scheme of the VUF6002 on the mouse models is disease condition-dependent. For example, an advanced stage of AD (17-month-old 3xTg-AD mice) may need a longer treatment scheme of VUF6002 for 3 months, while the mild stage of AD (8-month-old APP/PS1 mice) may need a relatively shorter VUF6002 treatment period for about 1 month.

Since AD is a lifelong disease for human, and thus the treatment of VUF6002 in humans may last for the rest of their life.

In an embodiment herein, the glucocorticoid (such as dexamethasone) is administrated at a dosage ranging from about 0.01 mg/kg to about 40 mg/kg; or from about 0.01 mg/kg to about 1 mg/kg; or from about 0.1 mg/kg to about 10 mg/kg in humans.

In an embodiment herein, the histamine H4 receptor antagonist or the glucocorticoid is administrated via a route selected from the group consisting of intraperitoneal administration, intracerebroventricular administration, oral administration, subcutaneous injection, or a combination thereof Intraperitoneal administration is the preferred administration route for it's a known route to cross the blood-brain barrier and affect microglial activation.

In an embodiment herein, the glucocorticoid is dexamethasone or any derivate thereof. Dexamethasone is an FDA-approved glucocorticoid that showed effectiveness in treating inflammation of the covid-19 patients. The present invention shows that dexamethasone can reduce Aβ level, hyperphosphorylated Tau and improve the cognitive function of the AD mice, which might outweigh those drugs in the clinical trials against AD that directly target the Aβ but didn't reveal cognitive improvement in the AD patients.

In an embodiment herein, the Aβ level both in the hippocampus and cortex may be reduced by administrating the glucocorticoid (e.g., dexamethasone or any derivate thereof). In some embodiments, the hyperphosphorylated Tau level may be reduced by administrating the glucocorticoid (e.g., dexamethasone or any derivate thereof). In some embodiments, both the Aβ level and the hyperphosphorylated Tau level may be reduced by administrating the glucocorticoid (e.g., dexamethasone or any derivate thereof).

The present application also relates to the following embodiments and various combinations thereof.

An embodiment of the invention relates to a method of therapy and/or prophylaxis of a neurodegenerative disease in a subject in need thereof, comprising administrating a histamine H4 receptor antagonist or a glucocorticoid, or their combination to the subject.

An embodiment of the invention relates to method of reversing cognitive and memory deficit in a subject in need thereof, comprising administrating a histamine H4 receptor antagonist or a glucocorticoid, or their combination to the subject.

An embodiment of the invention relates to method of decreasing Aβ level and hyperphosphorylated Tau in a subject in need thereof, comprising administrating a histamine H4 receptor antagonist and a glucocorticoid to the subject.

An embodiment of the invention relates to method of inhibiting microgliosis in hippocampus in a subject in need thereof, comprising administrating a histamine H4 receptor antagonist or a glucocorticoid, or their combination to the subject.

In the method according to any one of the preceding embodiments, the histamine H4 receptor antagonist can be selected from the group consisting of 1-[(5-chloro-1H-benzimidazol-2-yl)carbonyl]-4-methylpiperazine maleate (Formula I), 1-[(5-chloro-1H-indol-2-yl)carbonyl]-4-methylpiperazine (Formula II),

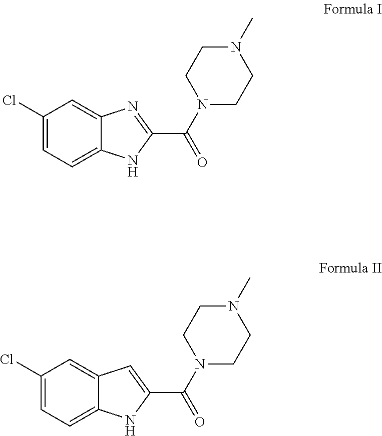 [SMILES: CN1CCN(C(=O)c2nc3cc(Cl)ccc3[nH]2)CC1 ; CN1CCN(C(=O)c2cc3cc(Cl)ccc3[nH]2)CC1]

or a pharmaceutically acceptable salt or derivate thereof.

In the method according to any one of the preceding embodiments, the neurodegenerative disease can be selected from the group consisting of Alzheimer's disease, Parkinson's disease, ischemic stroke, amyotrophic lateral sclerosis, and a combination thereof.

In the method according to any one of the preceding embodiments, the histamine H4 receptor antagonist can be administrated at a dosage of about 0.01 mg/kg to about 16 mg/kg on a daily basis.

In the method according to any one of the preceding embodiments, the histamine H4 receptor antagonist is administrated for from about 21 days to about 3 months.

In the method according to any one of the preceding embodiments, the histamine H4 receptor antagonist or the glucocorticoid may be administrated via a route selected from the group consisting of intraperitoneal administration, intracerebroventricular administration, oral administration, intravenous administration, intramuscular administration, subcutaneous injection, or a combination thereof.

In the method according to any one of the preceding embodiments, the glucocorticoid may be administrated at a dosage of about 0.01 mg/kg to about 40 mg/kg.

In the method according to any one of the preceding embodiments, the glucocorticoid may be dexamethasone or any derivate thereof.

In the method according to any one of the preceding embodiments, the Aβ level both in the hippocampus and cortex may be reduced.

### Animal Models of Alzheimer's Disease

In the examples herein, the following 3 types of AD mice are used to validate the beneficial effect of a histamine H4 receptor agonist, such as VUF6002 (Tocris, Hong Kong, China, https://www.tocris.com/): a sporadic AD mouse model induced by streptozocin, the 3xTg-AD mouse model (APP and Tau pathology), and the APP/PS1 mouse model (APP pathology).

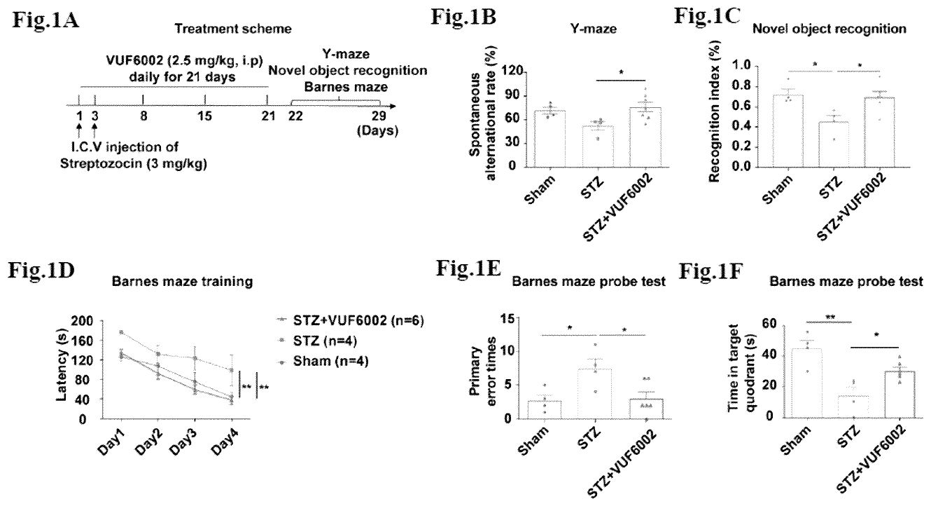

To induce sporadic Alzheimer's disease-like cognitive functional deficits, streptozocin (STZ) (Sigma-Aldrich, Hong Kong, China, https://www.sigmaaldrich.com/HK/en) is administrated via intracerebroventricular injections on adult male C57BL/6 mice (8 weeks old) (C57 black 6 mice, The Jackson Laboratory, Bar Harbor, Maine, USA, https://www.jax.org/). Adult male C57BL/6 mice are first anesthetized with ketamine (Alfasan International B.V, Woerden, Netherlands, https://www.alfasan.com/en/) (about 100 mg/kg) and xylazine (Alfasan International B.V, Woerden, Netherlands, https://www.alfasan.com/en/) (about 10 mg/kg) and placed in the stereotactic apparatus (RWD Life Science, Shenzhen, China, https://www.rwdstco.com/). A midline incision is made through the scalp, and the skin is laterally retracted to expose the skull surface. One microliter of freshly prepared streptozocin (about 60 mg/ml dissolved in 0.05M citrate buffer; pH=4.5) is injected bilaterally into the lateral ventricle at the following stereotactic coordinates relative to bregma: anteroposterior (AP)=±0.3 mm, mediolateral (ML)=±1.1 mm, and dorsoventral=+3.0 mm from the skull surface of both hemispheres using a 32G Hamilton syringe (RWD Life Science, Shenzhen, China, https://www.rwdstco.com/). Two administrations of STZ are performed on days 1 and 3, and cognitive functional deficits are developed at day 21 post-injection (see FIG. 1A for a detailed experimental paradigm). For the treatment paradigm of VUF6002, mice are received daily intraperitoneal injections of VUF6002 (2.5 mg/kg) for 21 days.

APP/PS1 mice (B6.Cg-Tg(APPswe,PSEN1dE9)85Dbo/Mmjax, The Jackson Laboratory, Bar Harbor, Maine, USA, https://www.jax.org/) show extracellular Aβ deposition at 6-month-old. This mouse model shows long-term memory deficits at around 7-month-old. Therefore, 7-month-old APP/PS1 mice receive daily intraperitoneal injections of VUF6002 (2.5 mg/kg) for 1 month.

The 7-month-old APP/PS1 mice are injected with dexamethasone (1 mg/kg) (Sigma-Aldrich, Hong Kong, China, https://www.sigmaaldrich.com/HK/en) for one month. The treatment using dexamethasone (1 mg/kg) can also be prolonged to two months from 6-month-old to 8-month-old of the APP/PS1 mice. At the 8-month-old, all mice participate the in neurobehavior test.

3xTg-AD mice (B6; 129-Tg (APPSwe, tauP301L) 1Lfa Psen1tm1Mpm/Mmjax, The Jackson Laboratory, Bar Harbor, Maine, USA, https://www.jax.org/) show extracellular Aβ deposition and increased level of phosphorylated Tau in the hippocampus at 12-month-old. For this, 3xTg-AD mice receive daily intraperitoneal injections of VUF6002 (2.5 mg/kg) for 3 months starting from 14-month-old, and age-matched C57BL/6 female mice serve as a control group.

All animal experiments are conducted by protocols approved by the Animal Research Ethics Sub-Committee at the City University of Hong Kong and Department of Health, HKSAR. Mice are provided with sufficient food and water ad libitum and maintained with a 12:12 h light-dark cycle.

### Animal Behavioral Assessment for Short-Term and Long-Term Memory Deficits in Alzheimer's Disease Mouse Model

### 1. Morris Water Maze Test

To assess the long-term spatial learning and memory functions of the mice, Morris Water Maze is performed in a round stainless-steel water tank with a diameter of 150 cm and a height of 60 cm, with a non-reflective interior surface to avoid any light reflections. On the test day, the water tank is filled with water made opaque by a non-toxic titanium dioxide with a water depth of 30 cm. The water temperature is maintained at 21±1° C. to avoid fatigue and hypothermia of the mice during the testing phase. The water tank is divided equally into four quadrants, and a circular PVC escape platform with a diameter of 12 cm is placed at the center of one quadrant submerged 1.5 cm below the water surface. Four different visual cues are placed on the wall next to the boundary of each quadrant within the water tank, and these cues remain in the same position throughout the acquisition training phase and the probe trial test.

Mice are given an acquisition training phase for 5 consecutive days. During each acquisition training day, the mice are first gently placed into one of the quadrants (except the target quadrant with the escape platform), and allowed to swim in the water tank for 90 seconds. During that period, the mice are allowed to orient their position in space with the navigation to the surrounding visual cues and the escape platform. The latency to reach the escape platform is recorded using ANY-maze software (Stoelting, Chicago, USA, https://stoeltingco.com/), and the representative tracking plot for each treatment group is generated using ANY-maze software (Stoelting, Chicago, USA, https://stoeltingco.com/). If the mice fail to locate the escape platform within 90 seconds, they are then guided to the escape platform and allowed to stay on the platform for 15 seconds. On each acquisition training day, the mice are trained four 4 times with different start positions in at least 30 minutes.

A probe trial test is performed one day after the 5-day acquisition training phase. During the probe trial test, the escape platform is removed from the water tank, and the mouse is allowed to explore freely in the water tank for 90 seconds. The time spent in the target quadrant and the total number of platform-site crossovers (i.e. total entry time to the site of the original escape platform) are recorded using ANY-maze software (Stoelting, Chicago, USA, https://stoeltingco.com/).

### 2. Barnes Maze Test

Barnes maze (diameter: 91 cm, height: 90 cm, 20 escape holes in the edge) is used to assess the long-term spatial memory of the mice. The Barnes maze is surrounded by 4 black PVC boards (120 cm in height, 10 cm away from the platform). A well-lit floodlight (500 W) is set above the Barnes maze. The floodlight is on during the whole process of the Barnes maze test to cause discomfort to mice. The escape box is put beneath one of the escape holes with a fixed location to let the mouse hide from the strong light.

The Barnes maze contains three trials: a habituation trial, a training trial, and a probe trial. In the habituation trial, mice could explore the maze freely within 2 minutes without environmental cues. If the mouse could not find the escape box, it would be guided to the escape box and stay inside for 30 seconds before going back to its home cage.

In the training stage, 4 visible cues are put on the PVC boards. The Barnes is divided into 4 equal quadrants based on the location of the cues. The quadrant that contains the escape box is defined as the target quadrant. The mouse is trained to locate the escape box according to the cues in this stage. The mouse is put inside the opaque box on the center of the platform for 10 seconds to make sure it did not know the direction. The Any-maze tracking system starts to record once the opaque box is lifted. In each trial, if the mouse could not enter the escape box within 3 minutes, it would be guided into the escape box and stay inside for 30 seconds. Each mouse would receive 3 times of training per day for 4 successive days. If the mouse enters the escape box within 3 minutes, the test will be ended and the test duration is referred to as the latency.

After 4 days of training, all mice participated in a probe test where the escape box is removed. The performance of each mouse in the probe test is video recorded. Each mouse is put inside the opaque box on the center of the platform for 10 seconds to make sure the mouse didn't know the direction. The test starts once the opaque is lifted. The mouse is allowed to explore the platform for 90 seconds. Mouse makes fewer primary error times (the times to visit the non-target holes) and spends more time in the target quadrant representing a better long-term spatial memory.

### 3. Y-Maze Test

A Y-shaped maze with 3 opaque arms (equal length) is utilized to determine the short-term spatial memory of mice. The three arms are labeled as zone A, zone B, and zone C. The maze also includes a hesitate zone in the center. Briefly, each mouse is placed at the center of the Y-maze and allowed to move freely for 8 minutes, and returns to the home cage. The instrument is sprayed with 70% ethanol to cover the smell and wiped thoroughly with tissue paper between each mouse. Only all four limbs of the mouse are inside an arm that counted as an entry. The visited zone list is recorded by the Any-maze tracking system. A spontaneous alternation is defined as a mouse entering 3 different arms of the maze consecutively (e.g., ABC, BCA, or CAB, but not BAB). The spontaneous alternation rate is calculated by dividing the number of alternations by the total number of entries minus 2.

### 4. Novel Object Recognition Test

A novel object recognition test is utilized to determine the recognition ability of mice. The test consists of a habituation trail, a training trial, and a test trial. In the habituation trial, each mouse could explore the empty open field box (50×50 cm) for 5 minutes and then return to the home cage. The instrument is sprayed with 70% ethanol to cover the smell and wiped thoroughly with tissue paper between each mouse. Twenty-four hours after habituation, mice then participate in a training trial. Two identical objects (opaque cylinder with 5 cm diameter and 10 cm height) are placed on a diagonal of the box at the same distance to the corner. Mice can explore the objects freely within 10 minutes. Exploring time is counted by the Any-maze system when the mouse head within 2 cm to the object. The training trial will be terminated if a mouse had explored the objects for an accumulative of 20 seconds or they use up 10 minutes time period. An hour later, one of the identical objects is replaced with a novel object (a 5×5×5 cm opaque cube). The mouse then performs a 5 minutes test trial, which is recorded by the Any-maze system. The instrument is sprayed with 70% ethanol to cover the smell and wiped thoroughly with tissue paper between each mouse.

Recognition index is defined as the exploring time of novel object/(the total exploring time of the novel object and the familiar object)×100% in the test trial.

When doing the behavior tests, the performer doesn't know about the genotypes and the treatment of the mice to exclude bias. The genotypes and the treatment of the mice is blinded by another colleague. After finishing the behavior test, the performer is unblinded and processes the data.

### Ouantification of Aβ, Phosphorylated Tau, and Microglia in 17-Month-Old 3xTg-AD Mice

The 17-month-old 3xTg-AD mice treated with VUF6002 and the 8-month-old APP/PS1 mice treated with dexamethasone are transcranial perfused with 4% paraformaldehyde (PFA), and the whole brain is harvested, postfixed, cryoprotected, and frozen in OCT compound (Sakura Finetek, Torrance, USA, https://www.sakuraus.com/). Forty-micrometer-thick coronal brain sections are blocked with 0.5% bovine serum albumin (BSA)/0.5% Triton X-100 (Sigma-Aldrich, Hong Kong, China, https://www.sigmaaldrich.com/HK/en) in PBS and incubated with primary antibodies against anti-6E10 (1:200; BioLegend, San Diego, USA, https://www.biolegend.com/), anti-AT8 (1:50; Thermo Fisher, Waltham, Massachusetts, USA, https://www.thermofisher.com/hk/en/home.html), and anti-IBA-1 (1:200, FUJIFILM Wako Chemicals, Richmond, USA, https://www.wakousa.com/) overnight at 4° C. After washing with ice-cold PBS three times, the cryosections are then incubated with corresponding secondary antibodies conjugated with Alexa Fluor 488 (1:200; Thermo Fisher, Waltham, Massachusetts, USA, https://www.thermofisher.com/hk/en/home.html) and Cy3 (1:200; Thermo Fisher, Waltham, Massachusetts, USA, https://www.thermofisher.com/hk/en/home.html) for 1 hour at room temperature. Images are captured at 20x magnification using Nikon A1HD25 confocal microscope equipped with a high-speed resonant scanner and a motorized stage, and stitched and maximal projected using NIS-Element's software (Nikon, Minato City, Tokyo, Japan, https://www.nikon.com/). For quantification, the contour of the hippocampal CA1 area or subiculum area is outlined manually and defined as regions of interest (ROIs) using NIS-Elements software (Nikon). The mean fluorescence intensity of the Aβ, phosphorylated Tau, or microglia within each ROI is determined using NIS-Elements software, and the area (in mm 2) of the hippocampal CA1 area of each section is measured. At least 4 coronal brain sections from each mouse with a similar brain location are quantified, and 3-4 mice per treatment group are analyzed. All the quantifications are performed blinded with genotypes and treatment.

### EXAMPLE 1

### VUF6002 as a Therapeutic Intervention to Treat Alzheimer's Disease

The inventors first evaluate the beneficial effects of VUF6002 on a sporadic AD mouse model induced by intra-cerebroventricular (ICV) injection of the streptozotocin (STZ) (Sigma-Aldrich, Hong Kong, China, https://www.sigmaaldrich.com/HK/en). In this model, the mice developed short-term and long-term memory deficits 21 days after ICV injection of STZ (FIG. 1A). As shown in FIG. 1A, adult C57BL/6 mice are intracerebroventricularly injected with STZ (3 mg/kg) at days 1 and 3, respectively, and then received intraperitoneal injections of VUF6002 at 2.5 mg/kg (or 1% Carboxymethyl cellulose solution as vehicle controls) for 21 consecutive days.

The animal models are firstly validated by performing Y-maze and novel object recognition test (FIG. 1B-1C), which on STZ-treated mice show severe impairment in short-term memory 21 days after STZ administration. The short-term spatial working memory is assessed by Y-maze spontaneous alternation test. As shown in FIG. 1B, STZ-treated mice show reduced alternation rate, which is completely reversed after VUF6002 treatment. The short-term recognition memory is assessed by novel object recognition test. As shown in FIG. 1C, VUF6002 treatment completely reverses the short-term memory deficits in STZ mice by increasing their ratio of time spent on the novel objects to a level comparable to the age-matched wild-type controls.

Also, long-term spatial memory is assessed by Barnes maze test on day 25 post injection. As shown in FIG. 1D, STZ-treated mice show significant impairment in long-term spatial memory as demonstrated by the increased latency to get into the hidden escape box in the Barnes maze test. Strikingly, VUF6002 completely reverses the STZ-induced long-term memory deficits in adult C57BL/6 mice.

The results of a probe trial test show that STZ-treated mice make more errors to find the target hole (FIG. 1E) and spend significantly less time on the target quadrant (FIG. 1F) compared with the vehicle-treated controls. In contrast, VUF6002 markedly increases the time spent in target quadrant and reduces the error to find the target hole in STZ-treated mice to a level comparable to the mice without STZ treatment. Mean±SEM. * P<0.05; ** P<0.01; two-way repeated measures ANOVA followed by Tukey post hoc test in FIG. 1D. One-way ANOVA followed by Tukey post hoc test in the other behavioral test.

In summary, treating the mice with VUF6002 (about 2.5 mg/kg) (Tocris, Hong Kong, China, https://www.tocris.com/) daily for 21 consecutive days completely reserves the short-term memory deficit (FIG. 1B-1C) and long-term spatial memory impairment induced by STZ administrations (FIG. 1D-1F), suggesting that VUF6002 may be effective for AD treatment.

### EXAMPLE 2

### Therapeutic Efficacy of VUF6002 in Reversing Cognitive and Memory Deficit in Mouse Models of AD

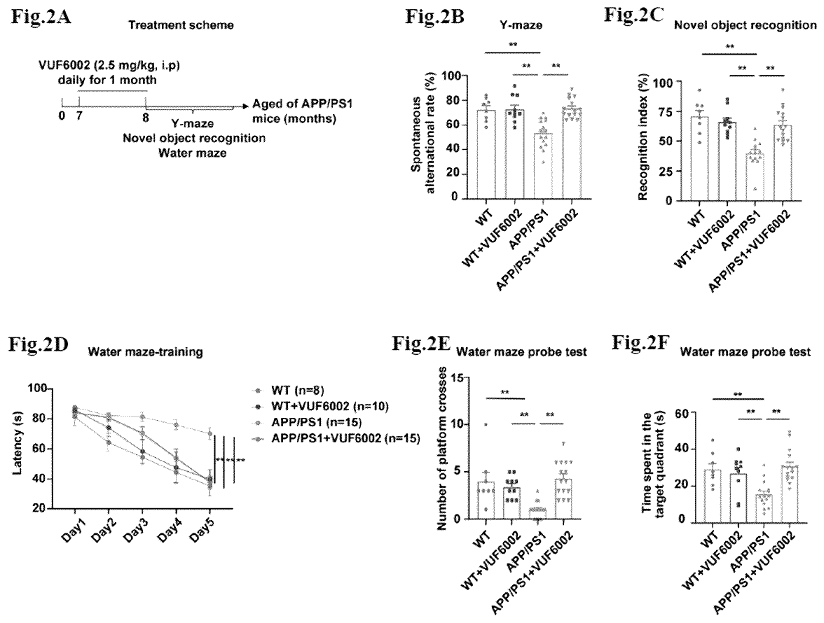

Next, the inventors evaluate the therapeutic efficacy of VUF6002 in reversing cognitive and memory deficits in two mouse models of AD. APP/PS1 mice could show promising memory deficit in neurobehavior test at around 7-month-old. Therefore, the APP/PS 1 mice are treated with VUF6002 at 2.5 mg/kg (or 1% Carboxymethyl cellulose (CMC) solution as vehicle controls) from 7-month-old to 8-month-old of mice. Short-term memory is evaluated by Y-maze and novel object recognition test, and long-term spatial memory is evaluated by Morris Water Maze test, respectively (FIG. 2A). Eight-months-old APP/PS1 mice demonstrate significant impairment in short-term memory (FIG. 2B-2C) and long-term spatial memory (FIG. 2D-2F).

Strikingly, treating the APP/PS1 mice with VUF6002 significantly elevates the spontaneous alternation rate in the Y-maze test (FIG. 2B) and recognition index in the novel object recognition test (FIG. 2C) compared with the vehicle-treated APP/PS1 mice. While vehicle-treated APP/PS1 mice show significant impairment in long-term spatial memory during the training phase of the Morris Water Maze test, a situation that is markedly improved after treating the mice with VUF6002 (FIG. 2D). In the probe test where the platform is removed, the VUF6002-treated APP/PS1 mice make more attempts to swim across the original location of the platform (FIG. 2E) and spend more time on searching the platform in the target quadrant (FIG. 2F), compared with the vehicle-treated APP/PS1 mice. Mean±SEM. * P<0.05; ** P<0.01; two-way repeated measures ANOVA followed by Tukey post hoc test in FIG. 2D. One-way ANOVA followed by Tukey post hoc test in the other behavioral test.

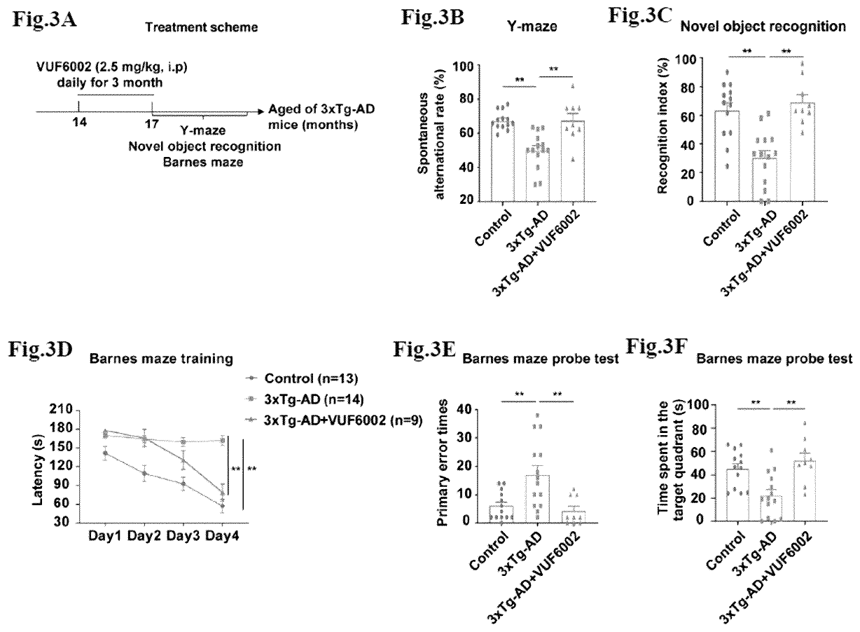

The inventors also evaluate the therapeutic efficacy of VUF6002 in improving memory deficit using a 3xTg-AD mouse model. The 3xTg-AD mice can manifest obvious extracellular Aβ deposition and memory deficits after 12-month-old. The 3xTg-AD mice are treated with VUF6002 at 2.5 mg/kg (or 1% Carboxymethyl cellulose (CMC) solution as vehicle controls) on a daily basis for about 3 months (from 14-month-old to 17-month-old) (FIG. 3A).

Animal behavioral assessment is performed on 17-month-old 3xTg-AD mice to assess short-term memory and long-term spatial memory. While vehicle-treated 17-month-old 3xTg-AD mice show significant impairment in short-term memory, VUF6002 completely reverse the memory deficit as demonstrated by improved performance on Y-maze and novel object recognition test (FIG. 3B-3C). Also, VUF6002-treated 3xTg-AD mice show a marked improvement during the training phase of Barnes Maze test (FIG. 3D).

In the probe test where the escape box is removed, VUF6002-treated 3xTg-AD mice make fewer errors to find the target hole and spend more time in the target quadrant when compared to vehicle-treated 3xTg-AD mice (FIG. 3E-3F). Mean±SEM. * P<0.05; ** P<0.01; two-way repeated measures ANOVA followed by Tukey post hoc test in FIG. 3D. One-way ANOVA followed by Tukey post hoc test in the other behavioral test.

These data indicate VUF6002 treatment rescues the short-term and long-term memory deficits as validated by 3 different AD mouse models.

### EXAMPLE 3

### Therapeutic Efficacy of VUF6002 in Decreasing Aβ Level and Hyperphosphorylated Tau in AD Mice via Immunomodulation

Whether VUF6002 could reduce the amount of Aβ level and the hyperphosphorylated Tau levels is determined as both Aβ level and hyperphosphorylated Tau levels are markedly increased in AD patients.

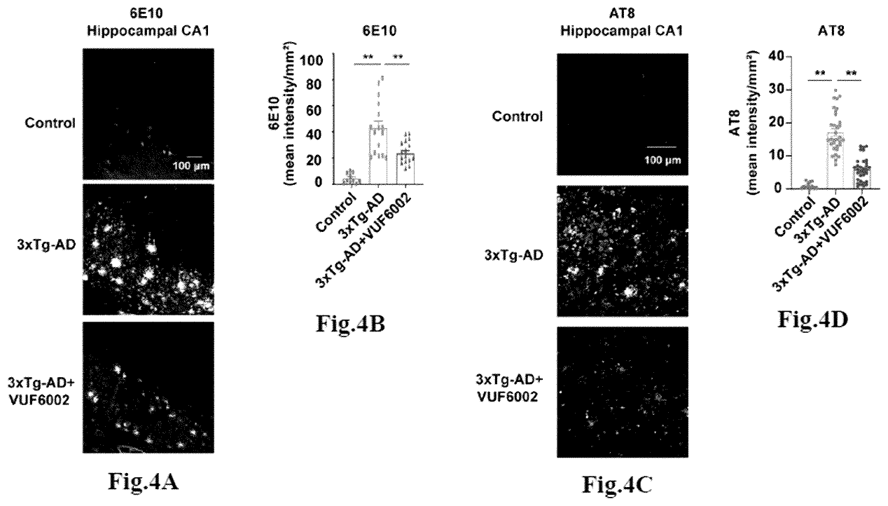

As shown in FIG. 4A, the immunoreactivity of anti-6E10 (both soluble and insoluble form of Aβ) increases dramatically in the CA1 region of hippocampus from vehicle-treated 17-month-old 3xTg-AD mice. In stark contrast, VUF6002 markedly reduces the immunoreactivity of anti-6E10 in the CA1 region of the hippocampus from 17-month-old 3xTg-AD mice. In age-matched wild-type mice, barely no immunoreactivity of human Aβ is detected in the entire hippocampus. The quantification of 6E10 fluorescence intensity is performed at CA1 region of hippocampus using NIS-EI-ements software (FIG. 4B). 1 hippocampal CA1 area (700 um*700 um) in one section and 4 coronal sections from each mouse are quantified.

The representative photomicrographs of hippocampus immunostained with anti-AT8 antibodies (which specifically immunolabelled intra- and extra-neuron hyperphosphorylated Tau (p-Tau) at Ser202 and Thr205) are shown in FIG. 4C, which reveal a dramatic increase in p-Tau immunoreactivity in the CA1 region of hippocampus from vehicle treated 17-month-old 3xTg-AD mice. However, VUF6002 markedly reduces the mean intensity of AT8 in 17-month-old 3xTg-AD mice. The quantification of AT8 fluorescence intensity is performed at CA1 region using NIS-Elements software (FIG. 4C). 2 hippocampal CA1 area (400 um*400 pm) in one section and 4 coronal sections from each mouse are quantified. Mean±SEM (n=3-4). * P<0.05; * P<0.01. One-way ANOVA followed by Tukey post hoc test in FIG. 4B, Student's t-test in FIG. 4D.

In view of the above, high immunoreactivity of Aβ in the hippocampus CA1 region is detected in vehicle-treated 17-month-old 3x Tg-AD mice; and the level of hyperphosphorylated Tau markedly increases in the hippocampus CA1 region of vehicle-treated 17-month-old 3x Tg-AD mice. Surprisingly, treating these mice with VUF6002 for 3 months strongly reduces the mean intensity of Aβ (FIG. 4A-4B) and hyperphosphorylated Tau (FIG. 4C-4D) in the hippocampus CA1 region. These results suggest that VUF6002 treatment could significantly reduce levels of the Aβ and hyperphosphorylated Tau in the hippocampus of 3xTg-AD mice.

### EXAMPLE 4

### Therapeutic Efficacy of VUF6002 in Inhibiting the Microgliosis in the Hippocampus in Mouse Models of AD

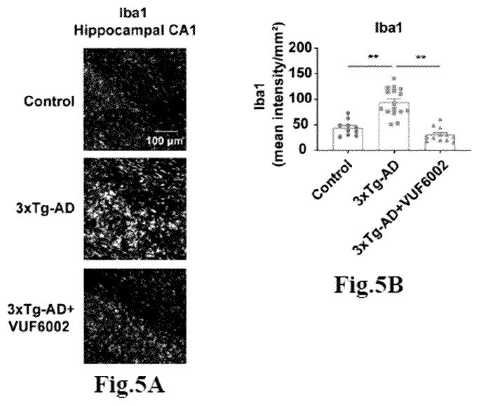

Neuroinflammation is believed to contribute to the pathologies of AD. The microglial density at the hippocampal CA1 area is evaluated using immunostaining of anti-IBA-1 primary antibodies on brain cryosections. At 17-month-old, a widespread microglial activation (as demonstrated by a significant increase in microglial density) in the hippocampal CA1 area in the 3xTg-AD mice compared to age-matched control mice is observed. As shown in FIG. 5A, no obvious activation of microglia is observed in age-matched wild-type mice, many Iba-1-positive activated microglia is clustered in the hippocampal CA1 region of 17-month-old 3xTg-AD mice, and VUF6002 markedly reduces the intensity of Iba-1-positive localized in the hippocampal CA1 region of 17-month-old 3xTg-AD mice. The quantification of Iba-1 fluorescence intensity is performed on the hippocampal CA1 region of 17-month-old 3xTg-AD mice. 4 coronal sections from each mouse and 12-16 hippocampal CA1 areas from one group are quantified. Mean±SEM (n=3-4). * P<0.05; ** P<0.01. One-way ANOVA followed by Tukey post hoc test in FIG. 5B. These results show that treating the 3xTg-AD mice with VUF6002 (2.5 mg/kg) significantly reduces the density of microglia in the same hippocampal region (FIG. 5B), suggesting a potential immunomodulatory effect of VUF6002 on ameliorating memory and cognitive functional deficits in the AD patients.

These examples show that a histamine receptor antagonist exhibits a prominent effect on rescuing the short-term and long-term memory deficits in a sporadic AD mouse model and two transgenic familiar AD mouse models as demonstrated by Y-maze, novel object recognition, Barnes maze, and Morris water maze test. Besides, it has been demonstrated that treating the 17-month-old 3xTg-AD mice with this histamine antagonist showed a significant reduction of the Aβ level, hyperphosphorylated Tau, and activated microglia in the hippocampal CA1 area. These results highlight the therapeutic effect of histamine antagonist in treating AD by reducing the AD pathologies and improving memory deficits in the AD mouse models.

### EXAMPLE 5

### Therapeutic Efficacy of Dexamethasone in Reversing Cognitive and Memory Deficit in a Mouse Model of AD

APP/PS1 mouse model develops extracellular Aβ deposition and reveals spatial memory deficit at 6 months old. In the first group, 7-month-old APP/PS1 mice are intraperitoneally injected with dexamethasone (1 mg/kg) for one month. In the second group, the mice are intraperitoneally injected with dexamethasone (1 mg/kg) for 2 months from 6-month-old to 8-month-old of the APP/PS1 mice. In the control group, the mice is injected with water. At the 8-month-old, all mice participated the in neurobehavior test.

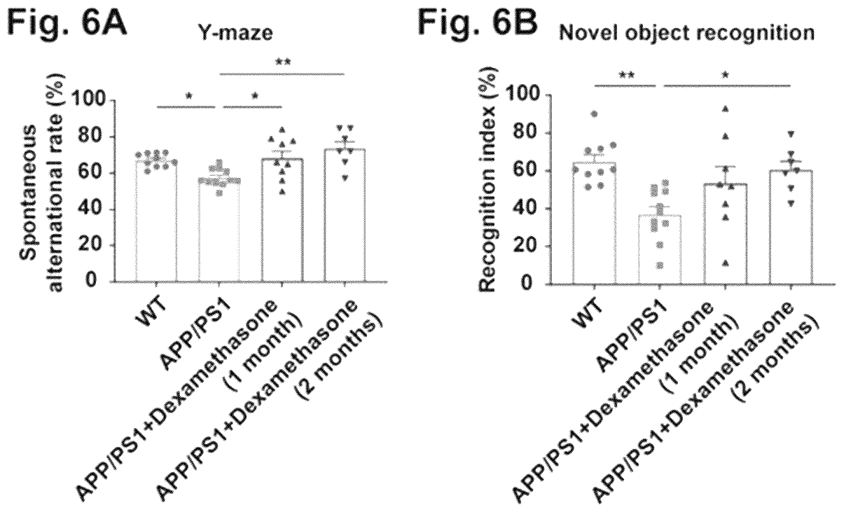

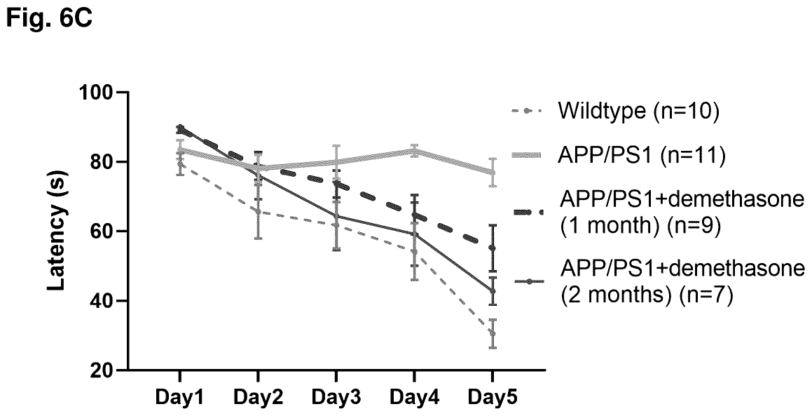

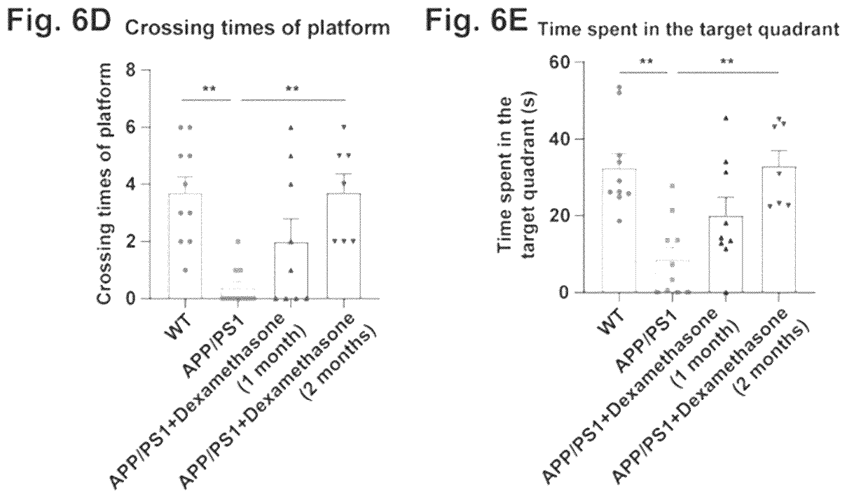

As shown in FIG. 6A, short-term spatial memory of the 8-month-old wildtype (WT) mice (n=10), vehicle-treated APP/PS1 mice (n=11), one month of Dexamethasone (1 mg/kg)-treated APP/PS1 mice (n=9), and two months of Dexamethasone (1 mg/kg)-treated APP/PS1 mice (n=7) is reflected by the spontaneous alternational rate in the Y-maze test. The results show that the 8-month-old vehicle-treated APP/PS1 mice reveal impaired short-term spatial working memory when compared to vehicle-treat wildtype mice in the Y-maze test. Two months of treatment of the dexamethasone prominently improves the short-term memory deficits in the APP/PS1 mice by reversing the spontaneous alternation rate to wild-type mice level (FIG. 6A).

The hippocampus-dependent recognition ability of the mice is accessed by the novel object recognition test, in which recognition index of different treatment groups are revealed by the percentage of time in exploring the novel object in the test. Vehicle-treated APP/PS1 mice reveal a recognition deficit reflected by a reduced time ratio in exploring the novel object. One month of treatment of the dexamethasone does not significantly improve the recognition deficits of the 8-month-old APP/PS1 mice. While two months of dexamethasone-treated APP/PS1 mice spend a higher ratio of time exploring the novel objects in comparison to the vehicle-treated APP/PS1 mice (FIG. 6B), indicating improved recognition ability of APP/PS1 mice by two months of treatment using dexamethasone.

The long-term spatial memory is assessed by the water maze test, in which the long-term spatial memory was revealed by the escape latency in the test. Vehicle-treated APP/PS1 mice reveal a higher escape latency to find the hidden platform compared to the age-matched wild-type mice, which suggests impaired long-term spatial learning memory of the mice. Two months treatment with dexamethasone reverses the long-term memory deficits of the APP/PS1 mice by reducing the latency to find the hidden platform relative to the 8-month-old APP/PS1 mice (FIG. 6C). While one-month treatment of the dexamethasone does not significantly reduce the latency compared to the vehicle-treated APP/PS1 mice.

In a probe test where the platform is removed, the number of platform crosses (FIG. 6D) and the time spent in the target quadrant (FIG. 6E) are recorded by the ANY-maze software. APP/PS1 mice with two months treatment of dexamethasone show conspicuously increased number of platform crosses (FIG. 6D) and time spent in the target quadrant (FIG. 6E) compared with the vehicle-treated APP/PS1 mice. These data indicate the two months treatment with dexamethasone can rescue the cognitive function deficits of 8-month-old APP/PS1 mice and has a better beneficial effect in comparison to the one-month treatment scheme. (Mean±SEM. * P<0.05; P<0.01; two-way repeated-measures ANOVA followed by Tukey post hoc test in (FIG. 6C). One-way ANOVA followed by Tukey post hoc test in (FIG. 6A-6B, and 6D-6E).

### EXAMPLE 6

### Therapeutic Efficacy of Dexamethasone in Decreasing Aβ Plaque and Hyperphosphorylated Tau in AD Mice

Whether or not dexamethasone could reduce the amount of Aβ plaque and hyperphosphorylated Tau in two AD mouse models is examined.

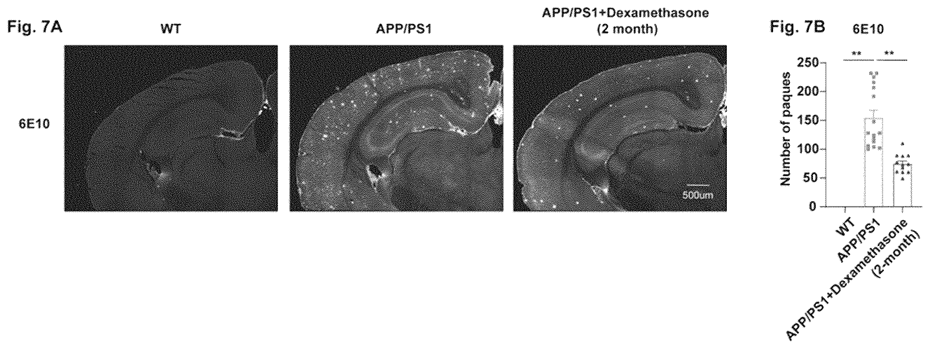

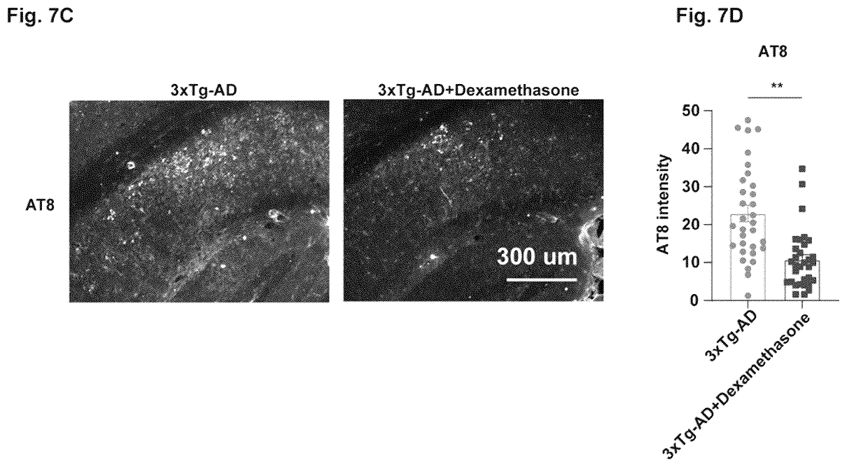

The soluble and insoluble forms of the Aβ are immunostained by anti-6E10 antibody in the brain of the 8-month-old wildtype (WT) mice (n=4), vehicle-treated APP/PS1 mice (n=4), and two months of Dexamethasone (1 mg/kg)-treated APP/PS1 mice (n=3). 4 sections from each mouse and 12-16 sections of each group are quantified. No Aβ plaque is detected in the vehicle-treated 8-month-old wild-type mice. In stark contrast, the Aβ plaques are scattered around the hippocampus and the cortex area of the 8-month-old vehicle-treated APP/PS1 mice (FIG. 7A).

The number of Aβ plaques is quantified in different treatment groups. (Mean±SEM. * P<0.05; ** P<0.01; One-way ANOVA followed by Tukey post hoc test in FIG. 7B). Two months treatment of the dexamethasone at 1 mg/kg remarkably reduces the number of Aβ plaques (51.6% reduction) in the 8-month-old APP/PS1 mice (FIG. 7B), indicating that dexamethasone can reduce the Aβ plaques in the brain of the 8-month-old APP/PS1 mice.

The inventors also use another AD mouse line, the 3xTg-AD mouse model that would develop the Tau pathology to validate dexamethasone against Tau pathology. The 3xTg-AD mice are treated with dexamethasone from 14-month-old to 17-month-old. The hyperphosphorylated Tau is immunostained by anti-AT8 antibody in the brain of the17-month-old 3xTg-AD mice (n=4), and 3xTg-AD with dexamethasone treatment (n=4). 8 sections from each mouse and 32 sections of each group are quantified. (Mean±SEM. * P<0.05; ** P<0.01; T-test)

As shown in FIGS. 7C and 7D, 3xTg-AD mice treated with dexamethasone strongly reduce the phosphorylated Tau intensity (53.3% reduction) in the hippocampal subiculum area of the 3xTg-AD mice, indicating that dexamethasone can reduce the hyperphosphorylated Tau in the brain of the 17-month-old 3xTg-AD mice.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

1. A method of therapy and/or prophylaxis of a neurodegenerative disease in a subject in need thereof, comprising administrating a histamine H4 receptor antagonist or a glucocorticoid, or their combination to the subject.

2. The method according to claim 1, wherein the histamine H4 receptor antagonist is selected from the group consisting of 1-[(5-chloro-1H-benzimidazol-2-yl)carbonyl]-4-methylpiperazine maleate (Formula I), 1-[(5-chloro-1H-indo1-2-yl)carbonyl]-4-methylpiperazine (Formula II), 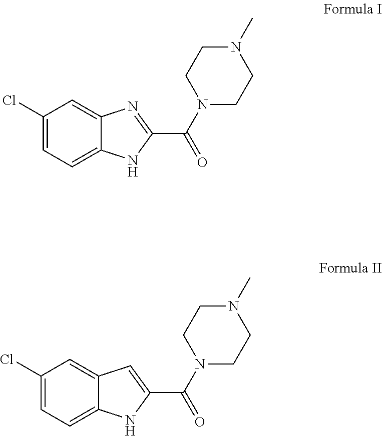 [SMILES: CN1CCN(C(=O)c2nc3cc(Cl)ccc3[nH]2)CC1 ; CN1CCN(C(=O)c2cc3cc(Cl)ccc3[nH]2)CC1]
or a pharmaceutically acceptable salt or derivate thereof, and wherein the glucocorticoid is dexamethasone or any derivate thereof.

3. The method according to claim 1, wherein the neurodegenerative disease is selected from the group consisting of Alzheimer's disease, Parkinson's disease, ischemic stroke, amyotrophic lateral sclerosis, and a combination thereof.

4. The method according to claim 1, wherein the histamine H4 receptor antagonist is administrated at a dosage of about 0.01 mg/kg to about 16 mg/kg on a daily basis, and wherein the glucocorticoid is administrated at a dosage of about 0.01 mg/kg to about 40 mg/kg.

5. The method according to claim 1, wherein the histamine H4 receptor antagonist is administrated for from about 21 days to about 3 months.

6. The method according to claim 1, wherein the histamine H4 receptor antagonist or the glucocorticoid is administrated via a route selected from the group consisting of intraperitoneal administration, intracerebroventricular administration, oral administration, intravenous administration, intramuscular administration, subcutaneous injection, or a combination thereof.

7. A method of decreasing Aβ level and hyperphosphorylated Tau in a subject in need thereof, comprising administrating a histamine H4 receptor antagonist and a glucocorticoid to the subject.

8. The method according to claim 7, wherein the Aβ level both in the hippocampus and cortex are reduced.

9. The method according to claim 1, wherein the histamine H4 receptor antagonist is selected from the group consisting of 1-[(5-chloro-1H-benzimidazol-2-yl)carbonyl]-4-methylpiperazine maleate (Formula I), 1-[(5-chloro-1H-indo1-2-yl)carbonyl]-4-methylpiperazine (Formula II), 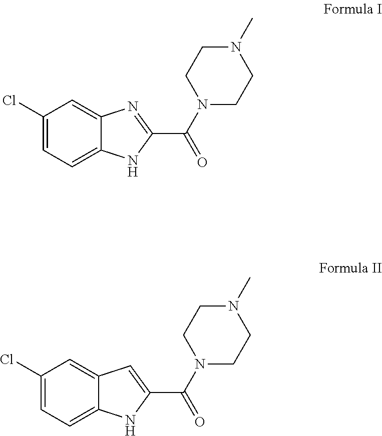 [SMILES: CN1CCN(C(=O)c2nc3cc(Cl)ccc3[nH]2)CC1 ; CN1CCN(C(=O)c2cc3cc(Cl)ccc3[nH]2)CC1]
or a pharmaceutically acceptable salt or derivate thereof, and wherein the glucocorticoid is dexamethasone or any derivate thereof.

10. The method according to claim 7, wherein the histamine H4 receptor antagonist is administrated at a dosage of about 0.01 mg/kg to about 16 mg/kg on a daily basis, and wherein the glucocorticoid is administrated at a dosage of about 0.01 mg/kg to about 40 mg/kg.

11. The method according to claim 7, wherein the histamine H4 receptor antagonist is administrated for from about 21 days to about 3 months.

12. The method according to claim 7, wherein the histamine H4 receptor antagonist or the glucocorticoid is administrated via a route selected from the group consisting of intraperitoneal administration, intracerebroventricular administration, oral administration, intravenous administration, intramuscular administration, subcutaneous injection, or a combination thereof.

13. A method of inhibiting microgliosis in hippocampus in a subject in need thereof, comprising administrating a histamine H4 receptor antagonist or a glucocorticoid, or their combination to the subject.

14. The method according to claim 13, wherein the histamine H4 receptor antagonist is selected from the group consisting of 1-[(5-chloro-1H-benzimidazol-2-yl)carbonyl]-4-methylpiperazine maleate (Formula I), 1-[(5-chloro-1H-indo1-2-yl)carbonyl]-4-methylpiperazine (Formula II), 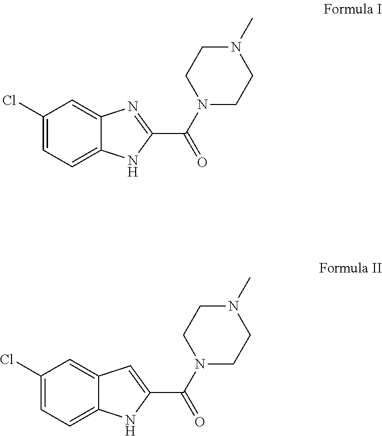 [SMILES: CN1CCN(C(=O)c2nc3cc(Cl)ccc3[nH]2)CC1 ; CN1CCN(C(=O)c2cc3cc(Cl)ccc3[nH]2)CC1]
or a pharmaceutically acceptable salt or derivate thereof, and wherein the glucocorticoid is dexamethasone or any derivate thereof.

15. The method according to claim 13, wherein the histamine H4 receptor antagonist is administrated at a dosage of about 0.01 mg/kg to about 16 mg/kg on a daily basis, and wherein the glucocorticoid is administrated at a dosage of about 0.01 mg/kg to about 40 mg/kg.

16. The method according to claim 13, wherein the histamine H4 receptor antagonist is administrated for from about 21 days to about 3 months.

17. The method according to claim 13, wherein the histamine H4 receptor antagonist or the glucocorticoid is administrated via a route selected from the group consisting of intraperitoneal administration, intracerebroventricular administration, oral administration, intravenous administration, intramuscular administration, subcutaneous injection, or a combination thereof.

18. A method of reversing cognitive and memory deficit in a subject in need thereof, comprising administrating a histamine H4 receptor antagonist or a glucocorticoid, or their combination to the subject.

19. The method according to claim 18, wherein the histamine H4 receptor antagonist is selected from the group consisting of 1-[(5-chloro-1H-benzimidazol-2-yl)carbonyl]-4-methylpiperazine maleate (Formula I), 1-[(5-chloro-1H-indo1-2-yl)carbonyl]-4-methylpiperazine (Formula II), 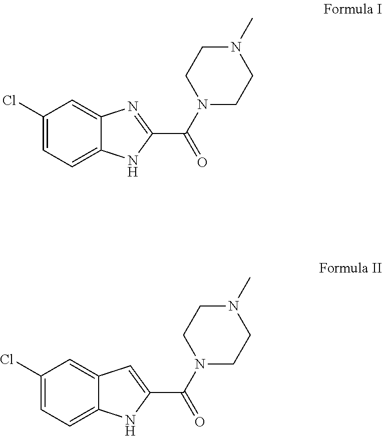 [SMILES: CN1CCN(C(=O)c2nc3cc(Cl)ccc3[nH]2)CC1 ; CN1CCN(C(=O)c2cc3cc(Cl)ccc3[nH]2)CC1]
or a pharmaceutically acceptable salt or derivate thereof, and wherein the glucocorticoid is dexamethasone or any derivate thereof.

20. The method according to claim 18, wherein the histamine H4 receptor antagonist is administrated at a dosage of about 0.01 mg/kg to about 16 mg/kg on a daily basis, and wherein the glucocorticoid is administrated at a dosage of about 0.01 mg/kg to about 40 mg/kg.
